(19) United States
(12) Patent Application Publication
(10) Pub. No.: US 2023/0405490 A1
(43) Pub. Date: Dec. 21, 2023

(54) IONIC LIQUID COMPOSITIONS FOR SELECTIVE REMOVAL OF SODIUM AND POTASSIUM FROM LITHIUM-CONTAINING AQUEOUS SOLUTIONS

(71) Applicant: UT-Battelle, LLC, Oak Ridge, TN (US)

(72) Inventors: Ilja Popovs, Knoxville, TN (US); Mary R. Healy, Alameda, CA (US); Bruce A. Moyer, Oak Ridge, TN (US)

(21) Appl. No.: 18/211,693

(22) Filed: Jun. 20, 2023

Related U.S. Application Data
(60) Provisional application No. 63/353,872, filed on Jun. 21, 2022.

(51) Int. Cl. B01D 11/04; C01D 15/04; C07C 311/48

(52) U.S. Cl. CPC B01D 11/0492; C07C 311/48; C01D 15/04

(57) ABSTRACT

A method for selectively removing sodium or potassium from an alkaline lithium-containing aqueous solution, the method comprising: (i) contacting the alkaline lithium-containing aqueous solution with a hydrophobic solution comprising: (a) an aqueous-insoluble hydrophobic solvent, (b) a protic ionic liquid of the formula X⁻Y⁺, wherein X⁻ is a conjugate base of a superacid and Y⁺ is a protonated cation, and (c) at least one of lipophilic sodium-selective and potassium-selective complexing ligands, wherein the contacting step results in selective complexation with and removal of sodium and/or potassium ions from the lithium-containing aqueous solution into the hydrophobic solution along with simultaneous abstraction of a proton from Y⁺ to form Y; and (ii) separating the aqueous solution from the hydrophobic solution, wherein, in some embodiments, X⁻ is a bis(sulfonyl)imide and Y⁺ is a protic ammonium species. The method may further include stripping sodium and potassium ions from the hydrophobic solution and regenerating Y⁺.

### CROSS REFERENCE TO RELATED APPLICATION

The present application claims benefit of U.S. Provisional Application No. 63/353,872, filed on Jun. 21, 2022, all of the contents of which are incorporated herein by reference.

### STATEMENT REGARDING FEDERALLY SPONSORED RESEARCH

This invention was made with government support under Prime Contract No. DE-AC05-00OR22725 awarded by the U.S. Department of Energy. The government has certain rights in the invention.

### FIELD OF THE INVENTION

The present invention generally relates to materials and methods for removing impurities from lithium-containing solutions for the purpose of obtaining lithium salts in pure form. The present invention more particularly relates to methods of selectively removing sodium and potassium from lithium-containing solutions to obtain lithium salts in pure form.

### BACKGROUND OF THE INVENTION

Methods for the isolation and purification of lithium from lithium-containing solutions, including terrestrial brines, geothermal brines, and others, are continually being sought but with limited success. The conventional methods are generally energy intensive and time consuming. For example, thermal or solar evaporation generally requires heating by combustion of fossil fuels or reliance on solar radiation and wind, any of which typically requires 18-24 months to produce a final lithium salt product. Moreover, water consumption and water management have become issues of significant concern, particularly since a large number of evaporation ponds and lithium production sites are located in arid regions of the world, such as the Atacama dessert.

As lithium has gained importance as an element for use in various applications, there are continuing efforts to develop less costly and more efficient methods for the recovery of lithium. In particular, there have been continuing efforts in the use of solvent extraction techniques for the selective removal of lithium from lithium-containing solutions. However, such techniques generally rely on a limited number of lithium complexing molecules, which are costly and limited in their selectivity for lithium over other cations typically present, such as sodium and potassium.

Thus, there would be a significant advantage in a process that could bypass the shortcomings of the conventional art and not rely on the selective extraction of lithium from aqueous solutions containing high concentrations of other ions. Such a process, which has thus far remained elusive, would provide a more cost-effective and economical alternative for isolating and purifying lithium than the currently available technology.

### SUMMARY

The present invention overcomes the above obstacles of the conventional art by bypassing the need to selectively extract lithium from lithium-containing solutions in an effort to isolate and purify the lithium. Instead of selectively removing lithium, the present invention relies on the selective removal of sodium and/or potassium from the lithium-containing solution as the primary means for further isolating and purifying the lithium. The invention achieves this by contacting an aqueous lithium-containing solution with a hydrophobic solution containing: (a) a hydrophobic solvent, (b) a lipophilic protic ionic liquid containing a conjugate base of a superacid, and (c) a lipophilic sodium-selective or potassium-selective complexing ligand, with the end result of selectively removing sodium and/or potassium ions from the aqueous lithium-containing solution and transferring them to the hydrophobic solution.

The protic ionic liquid can be represented by the formula of the formula X⁻Y⁺, wherein X⁻ is a conjugate base of a superacid and Y⁺ is a protonated cation. In particular embodiments, the protic ionic liquid has the following formula:

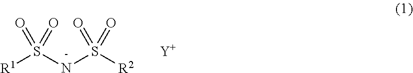

wherein R¹ and R² are independently selected from non-fluorinated hydrocarbon groups containing at least 3 carbon atoms with optional presence of a single —O— linker; and Y⁺ is a protonated cation.

In some embodiments of Formula (1), Y⁺ is an ammonium species. Since Y⁺ is protonated, the ammonium species necessarily contains at least one hydrogen atom (proton) attached to the nitrogen atom. In some embodiments, the ammonium species has the following structure:

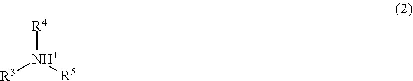

wherein R³, R⁴, and R⁵ are independently selected from hydrocarbon groups containing 3-30 carbon atoms.

In other embodiments, Y⁺ is a heteroaromatic ring containing at least one nitrogen atom, at least one of which is positively-charged. The heteroaromatic ring may be selected from, for example, pyridinium, imidazolium, piperidinium, and pyrrolidinium.

In particular embodiments, the ionic liquid has the following formula:

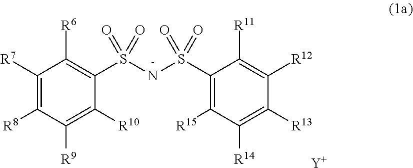

wherein: R⁶, R⁷, R⁸, R⁹, R¹⁰, R¹¹, R¹², R¹³, R¹⁴, and R¹⁵ are independently selected from H atom and non-fluorinated hydrocarbon groups having at least 1, 2, 3, 4, 5, or 6 carbon atoms with optional presence of a single —O— linker, wherein at least one of R⁶, R⁷, R⁸, R⁹, R¹⁰, R¹¹, R¹², R¹³, R¹⁴, and R¹⁵ is a non-fluorinated hydrocarbon group containing 1, 2, 3, 4, 5, or 6 carbon atoms; and Y⁺ is a protonated cation, such as any of the protonated cations described in the present disclosure, including any of the ammonium species described in the present disclosure. In separate or further embodiments, at least two, three, four, or more of R⁶, R⁷, R⁸, R⁹, R¹⁰, R¹¹, R¹², R¹³, R¹⁴, and R¹⁵ are non-fluorinated hydrocarbon groups containing at least 1, 2, 3, 4, 5, or 6 carbon atoms.

The method for selectively removing sodium or potassium from an alkaline lithium-containing aqueous solution (e.g., a lithium-containing brine solution) preferably includes the following steps: (i) contacting the alkaline lithium-containing aqueous solution with a hydrophobic solution comprising: (a) a hydrophobic solvent, (b) a protic ionic liquid of the formula X⁻Y⁺, wherein X⁻ is a conjugate base of a superacid and Y⁺ is a protonated cation, which may correspond to any of the protic ionic liquids described above and elsewhere in the present disclosure, and (c) at least one of lipophilic sodium-selective and potassium-selective complexing ligands, wherein the contacting step results in selective complexation with and transfer of sodium and/or potassium ions from the lithium-containing aqueous solution into the hydrophobic solution along with simultaneous abstraction of a proton from Y⁺ to form Y; and (ii) separating the aqueous solution from the hydrophobic solution. In some embodiments, the method further includes: stripping sodium and potassium ions from the hydrophobic solution by treating the hydrophobic solution with an aqueous stripping solution containing an acid, wherein the stripping process simultaneously reprotonates Y to regenerate the ionic liquid X⁻Y⁺ in the hydrophobic solution. In some embodiments, the lipophilic sodium-selective complexing ligand is a lipophilic crown ether molecule and is present in the hydrophobic solution. In further or separate embodiments, the lipophilic potassium-selective complexing ligand is a calixarene molecule and is present in the hydrophobic solution. In some embodiments, both the lipophilic sodium-selective and lipophilic potassium-selective complexing ligands are present in the hydrophobic solution.

The lithium purification process described herein is advantageously and cost-efficient while at the same time capable of removing a substantial portion or all of the sodium and potassium contaminants from an aqueous source containing lithium. The process also advantageously does not rely on thermal or solar evaporation, nor does the process rely on sorbent methods, such as columns packed with layered lithium aluminate sorbents. The process described herein circumvents the substantial drawbacks associated with the conventional methods, and instead relies on a straight-forward and cost-effective method of removing sodium and potassium ions for isolating and purifying lithium in lithium-containing solutions.

### BRIEF DESCRIPTION OF THE DRAWINGS

FIG. 1 is a schematic of a solvent extraction process for the removal of Na⁺ and K⁺ ions from a lithium-containing solution using an exemplary class of lipophilic protic ionic liquids combined with L1 or L2 (lipophilic sodium or potassium complexing agents, respectively).

FIG. 2 is a graph showing potassium distribution values for IL-1 and L2+IL-1 separation systems (structures of these species given in the Examples section).

FIG. 3 is a graph showing solvent extraction ligand metal loading and extraction efficiency expressed as percent metal extracted for different solvents in the presence and absence of co-extractant.

FIG. 4 is a graph showing distribution values of metal ions during the extraction with a protic ionic liquid (IL-1) alone in various solvents.

FIG. 5 is a graph showing distribution values of metal ions during the extraction with a protic ionic liquid (IL-1) in the presence of L2 in various solvents.

### DETAILED DESCRIPTION

As used herein, the term “hydrocarbon group” (also denoted by the group R) is defined as a chemical group composed of at least of carbon and hydrogen. In some embodiments, the hydrocarbon group is composed solely of carbon and hydrogen. In other embodiments, the hydrocarbon group may (i.e., optionally) be substituted with one or more fluorine atoms to result in partial or complete fluorination of the hydrocarbon group. In other embodiments, as further discussed below, the hydrocarbon group may be substituted with one or more other heteroatoms (e.g., O, N, or S) or heteroatom-containing groups.

The hydrocarbon group (R) typically contains 1-30 carbon atoms, but may contain over 30 carbon atoms, such as 40, 50, 60, 70, or 80 carbon atoms. In different embodiments, one or more of the hydrocarbon groups may contain, for example, 1, 2, 3, 4, 5, 6, 7, 8, 9, 10, 12, 15, 18, 20, 22, 24, 26, 28, or 30 carbon atoms, or a number of carbon atoms within a particular range bounded by any two of the foregoing carbon numbers (e.g., 1-80, 1-50, 3-80, 3-50, 1-30, 2-30, 3-30, 4-30, 6-30, 8-30, 10-30, 12-30, 1-20, 6-20, 8-20, 10-20, or 12-20 carbon atoms). Hydrocarbon groups in different compounds described herein, or in different positions of a compound, may possess the same or different number (or preferred range thereof) of carbon atoms in order to independently adjust or optimize such properties as the complexing ability, extracting (extraction affinity) ability, or selectivity ability.

In a first set of embodiments, the hydrocarbon group (R) is a saturated and straight-chained group, i.e., a straight-chained (linear) alkyl group. Some examples of straight-chained alkyl groups include methyl, ethyl, n-propyl, n-butyl, n-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-eicosyl, n-docosyl, n-tetracosyl, n-hexacosyl, n-octacosyl, and n-triacontyl groups.

In a second set of embodiments, the hydrocarbon group (R) is saturated and branched, i.e., a branched alkyl group. Some examples of branched alkyl groups include isopropyl (2-propyl), isobutyl (2-methylprop-1-yl), sec-butyl (2-butyl), t-butyl (1,1-dimethylethyl-1-yl), 2-pentyl, 3-pentyl, 2-methylbut-1-yl, isopentyl (3-methylbut-1-yl), 1,2-dimethylprop-1-yl, 1,1-dimethylprop-1-yl, neopentyl (2,2-dimethylprop-1-yl), 2-hexyl, 3-hexyl, 2-methylpent-1-yl, 3-methylpent-1-yl, isohexyl (4-methylpent-1-yl), 1,1-dimethylbut-1-yl, 1,2-dimethylbut-1-yl, 2,2-dimethylbut-1-yl, 2,3-dimethylbut-1-yl, 3,3-dimethylbut-1-yl, 1,1,2-trimethylprop-1-yl, 1,2,2-trimethylprop-1-yl, isoheptyl, isooctyl, and the numerous other branched alkyl groups having up to 20, 30, or more carbon atoms, wherein the “1-yl” suffix represents the point of attachment of the group.

In a third set of embodiments, the hydrocarbon group (R) is saturated and cyclic, i.e., a cycloalkyl group. Some examples of cycloalkyl groups include cyclopropyl, cyclobutyl, cyclopentyl, cyclohexyl, cycloheptyl, and cyclooctyl groups. The cycloalkyl group can also be a polycyclic (e.g., bicyclic) group by either possessing a bond between two ring groups (e.g., dicyclohexyl) or a shared (i.e., fused) side (e.g., decalin and norbornane).

In a fourth set of embodiments, the hydrocarbon group (R) is unsaturated and straight-chained, i.e., a straight-chained (linear) olefinic or alkenyl group. The unsaturation occurs by the presence of one or more carbon-carbon double bonds and/or one or more carbon-carbon triple bonds. Some examples of straight-chained olefinic groups include vinyl, propen-1-yl (allyl), 3-buten-1-yl (CH₂═CH—CH₂—CH₂—), 2-buten-1-yl (CH₂—CH═CH—CH₂—), butadienyl, 4-penten-1-yl, 3-penten-1-yl, 2-penten-1-yl, 2,4-pentadien-1-yl, 5-hexen-1-yl, 4-hexen-1-yl, 3-hexen-1-yl, 3,5-hexadien-1-yl, 1,3,5-hexatrien-1-yl, 6-hepten-1-yl, ethynyl, propargyl (2-propynyl), 3-butynyl, and the numerous other straight-chained alkenyl or alkynyl groups having up to 20, 30, or more carbon atoms.

In a fifth set of embodiments, the hydrocarbon group (R) is unsaturated and branched, i.e., a branched olefinic or alkenyl group. Some examples of branched olefinic groups include propen-2-yl (CH₂═C.—CH₃), 1-buten-2-yl (CH₂═C.—CH₂—CH₃), 1-buten-3-yl (CH₂═CH—CH.—CH₃), 1-propen-2-methyl-3-yl (CH₂═C(CH₃)—CH₂—), 1-penten-4-yl, 1-penten-3-yl, 1-penten-2-yl, 2-penten-2-yl, 2-penten-3-yl, 2-penten-4-yl, and 1,4-pentadien-3-yl, and the numerous other branched alkenyl groups having up to 20, 30, or more carbon atoms, wherein the dot in any of the foregoing groups indicates a point of attachment.

In a sixth set of embodiments, the hydrocarbon group (R) is unsaturated and cyclic, i.e., a cycloalkenyl group. The unsaturated cyclic group may be aromatic or aliphatic. Some examples of unsaturated cyclic hydrocarbon groups include cyclopropenyl, cyclobutenyl, cyclopentenyl, cyclopentadienyl, cyclohexenyl, cyclohexadienyl, phenyl, benzyl, cycloheptenyl, cycloheptadienyl, cyclooctenyl, cyclooctadienyl, and cyclooctatetraenyl groups. The unsaturated cyclic hydrocarbon group may or may not also be a polycyclic group (such as a bicyclic or tricyclic polyaromatic group) by either possessing a bond between two of the ring groups (e.g., biphenyl) or a shared (i.e., fused) side, as in naphthalene, anthracene, phenanthrene, phenalene, or indene fused ring systems.

As indicated earlier above, any of the hydrocarbon groups described above may be substituted with one or more fluorine atoms. As an example, an n-octyl group may be substituted with a single fluorine atom to result in, for example, a 7-fluorooctyl or 8-fluorooctyl group, or substituted with two or more fluorine atoms to result in, for example, 7,8-difluorooctyl, 8,8-difluorooctyl, 8,8,8-trifluorooctyl, or perfluorooctyl group. Any of the hydrocarbon groups described herein may contain a single ether (—O—) or thioether (—S—) linkage connecting between carbon atoms in the hydrocarbon group. An example of a hydrocarbon group containing a single ether or thioether group is —(CH₂)₂—X—(CH₂)₇ CH₃, wherein X represents O or S.

As indicated earlier above, any of the hydrocarbon groups described above may be substituted with one or more heteroatom-containing groups. Some examples of heteroatom-containing groups include —OH, —OR″, —NH₂, —NHR″, —NR″₂, —NO₂, —SR″, —SO₂R″, —SO₂NR″₂, —C(O)R″, —C(O)OR″, —C(O)NH₂, —C(O)NHR″, —C(O)NR″₂, —C(S)OR″, —C(O)SR″, —C(S)NH₂, —C(S)NHR″, and —C(S)NR″₂ groups, wherein R″ groups are independently selected from alkyl groups containing 1-20 carbon atoms. In some embodiments, any one or more heteroatom-containing groups are excluded from the hydrocarbon groups. In some embodiments, the hydrocarbon group(s) is/are composed of carbon and hydrogen with the exception that they may (i.e., optionally) include a single ether (—O—) group.

In one aspect, the present disclosure is directed to protic ionic liquids of the formula X⁻Y⁺, wherein X⁻ is a conjugate base of a superacid and Y⁺ is a protonated cation. X⁻ is a conjugate base of any of the superacids known in the art. In addition to being a conjugate base of a superacid, X⁻ is preferably also a delocalized anion that has a non-nucleophilic and non-coordinating behavior. Some examples of anions suitable as X⁻ include the bis(sulfonyl)imides, sulfonates (i.e., RSO₃⁻, e.g., trifluoromethanesulfonate), sulfates (ROSO₃⁻), sulfonyl carboxamides (RSO₂NCOR⁻), carborane anions, and dicarbolide anions (e.g., cobalt bis(dicarbolide) anion). Y⁺ can be any protonated cation, including cations in which a proton is located on a nitrogen atom, phosphorus atom, or sulfur atom (i.e., ammonium, phosphonium, and sulfonium cations, respectively). X⁻ and Y⁺ should each be sufficiently lipophilic to the extent that the resulting ionic liquid (X⁻Y⁺) is soluble in a non-polar solvent substantially or completely immiscible with water, typically a hydrocarbon solvent.

In particular embodiments, the protic ionic liquid has a bis(sulfonyl)imide structure within the following generic structure:

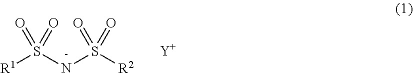

In Formula (1) above, R¹ and R² may be the same or different and are independently selected from non-fluorinated hydrocarbon groups containing at least 3, 4, 5, or 6 carbon atoms (or more particularly, 3-80, 3-50, or 3-30 carbon atoms) with optional presence of a single —O— linker. R¹ and R² may be independently selected from any of the hydrocarbon groups (R) described earlier above. In different embodiments, R¹ and R² may independently contain 3, 4, 5, 6, 7, 8, 9, 10, 12, 14, 16, 18, 20, 22, 24, 26, 28, 30, 40, 50, 60, 70, or 80 carbon atoms or a number of carbon atoms within a range bounded by any two of the foregoing values, e.g., 3-80, 3-70, 3-60, 3-50, 3-40, 3-30, 3-20, 3-16, 3-12, 4-30, 4-20, 4-16, 4-12, 6-30, 6-20, 6-16, 6-12, 8-30, 8-20, 8-16, or 8-12. In some embodiments, an —O— linker (or any heteroatom) is absent from R¹ and R², in which case R¹ and R² are composed of carbon and hydrogen only. In some embodiments, R¹ and R² are independently selected from linear or branched alkyl or alkenyl groups independently containing any of the number of carbon atoms or ranges thereof provided above. In some embodiments, R¹ and R² are independently selected from cycloalkyl or aromatic rings, such as cyclopentyl, cyclohexyl, and phenyl rings, wherein one or both rings may or may not be substituted with one or more linear or branched alkyl or alkenyl groups containing 1-30, 1-20, 1-12, 1-6, 3-30, 3-20, 3-12, or 3-6 carbon atoms.

Y⁺ in X⁻Y⁺ or Formula (1) can be any protonated cation, including cations in which a proton is located on a nitrogen atom, phosphorus atom, or sulfur atom (i.e., ammonium, phosphonium, and sulfonium cations, respectively), provided that the resulting ionic liquid is soluble in a non-polar solvent substantially or completely immiscible with water, typically a hydrocarbon solvent.

In particular embodiments, the protic ionic liquid of Formula (1) has a bis(phenylsulfonyl)imide structure, such as shown by the following structure:

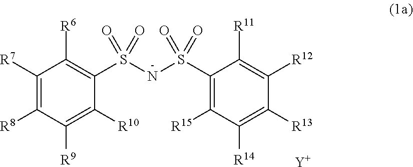

In Formula (1a), R⁶, R⁷, R⁸, R⁹, R¹⁰, R¹¹, R¹², R¹³, R¹⁴, and R¹⁵ are independently selected from hydrogen (H) atom and non-fluorinated hydrocarbon groups having 1-30, 1-20, 1-12, 3-30, 3-20, or 3-12 carbon atoms with optional presence of a single —O— linker, wherein at least one of R⁶, R⁷, R⁸, R⁹, R¹⁰, R¹¹, R¹², R¹³, R¹⁴, and R¹⁵ is a non-fluorinated hydrocarbon group containing 1-30 or 1-20 carbon atoms. In some embodiments, precisely or at least two of R⁶, R⁷, R⁸, R⁹, R¹⁰, R¹¹, R¹², R¹³, R¹⁴, and R¹⁵ are independently selected from non-fluorinated hydrocarbon groups containing 1-30, 1-20, 1-12, 3-30, 3-20, or 3-12 carbon atoms. In other embodiments, precisely or at least three, four, five, or six of R⁶, R⁷, R⁸, R⁹, R¹⁰, R¹¹, R¹², R¹³, R^{14,} and R¹⁵ are independently selected from non-fluorinated hydrocarbon groups containing 1-30, 1-20, 1-12, 3-30, 3-20, or 3-12 carbon atoms. Y⁺ in Formula (1a) can be any protonated cation, including cations in which a proton is located on a nitrogen atom, phosphorus atom, or sulfur atom (i.e., ammonium, phosphonium, and sulfonium cations, respectively), provided that the resulting ionic liquid according to Formula (1a) is soluble in a non-polar solvent substantially or completely immiscible with water, typically a hydrocarbon solvent.

In particular embodiments of X⁻Y⁺, Formula (1), or Formula (1a), Y⁺ is an ammonium species. Since Y⁺ is protonated, the ammonium species necessarily contains at least one hydrogen atom attached to the nitrogen atom. In different embodiments, the ammonium species contains one, two, three, or four hydrogen atoms attached to the nitrogen atom, with remaining positions on the nitrogen atom, if any, being hydrocarbon groups, preferably non-fluorinated. In typical embodiments, the ammonium species contains only one hydrogen atom attached to the nitrogen atom, with the remaining three groups being hydrocarbon groups.

In some embodiments, the ammonium species has the following formula:

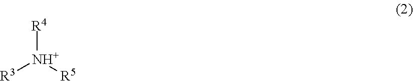

In Formula (2), R³, R⁴, and R⁵ are independently selected from hydrogen atom and hydrocarbon groups containing 3-30 carbon atoms. In some embodiments, R³, R⁴, and R⁵ are independently selected from hydrocarbon groups containing 3-30 carbon atoms. R³, R⁴, and R⁵ may be independently selected from any of the hydrocarbon groups (R) described earlier above containing 3-30 carbon atoms. In different embodiments, R³, R⁴, and R⁵ may independently contain 3, 4, 5, 6, 7, 8, 9, 10, 12, 14, 16, 18, 20, 22, 24, 26, 28, or 30 carbon atoms or a number of carbon atoms within a range bounded by any two of the foregoing values, e.g., 3-30, 3-20, 3-16, 3-12, 4-30, 4-20, 4-16, 4-12, 6-30, 6-20, 6-16, 6-12, 8-30, 8-20, 8-16, or 8-12. In some embodiments, R³, R⁴, and/or R⁵ are composed of carbon and hydrogen only. In some embodiments, R³, R⁴, and/or R⁵ are independently selected from linear or branched alkyl or alkenyl groups independently containing any of the number of carbon atoms or ranges thereof provided above. In some embodiments, R³, R⁴, and R⁵ are independently selected from cycloalkyl or aromatic rings, such as cyclopentyl, cyclohexyl, and phenyl rings, wherein one or both rings may or may not be substituted with one or more linear or branched alkyl or alkenyl groups containing 1-20, 1-12, 1-6, 1-3, 3-20, 3-12, or 3-6 carbon atoms.

In other particular embodiments of Formula (1) or (1a), Y⁺ is a heterocyclic (or more particularly, heteroaromatic) ring containing at least one nitrogen atom, at least one of which is positively-charged. The heterocyclic ring may be, for example, pyridinium, imidazolium, piperidinium, or pyrrolidinium.

The protic ionic liquids according to Formula (1) or (1a) can be synthesized by means well known in the art. For example, a bis(phenylsulfonyl)imide molecule according to Formula (1a), can be produced by a base-mediated reaction between a corresponding sulfonamide and sulfonylchloride in an appropriate solvent, such as water, acetonitrile, or tetrahydrofuran.

In another aspect, the present disclosure is directed to lipophilic sodium-selective and potassium-selective complexing ligands. By being selective, the foregoing ligands selectively complex with sodium or potassium ions over lithium ions.

The lipophilic sodium-selective ligand may be, for example, a lipophilic crown ether molecule. The lipophilic crown ether molecule may have the following general formula:

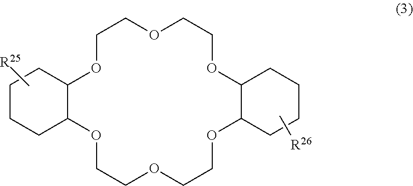

In Formula (3), R²⁵ and R²⁶ are the same or different and are independently selected from hydrocarbon groups (or more specifically, linear or branched alkyl groups) containing 1-20 carbon atoms, or more particularly, 1-12, 1-6, 1-4, 1-3, 2-20, 2-12, 2-6, 2-4, 3-20, 3-12, or 3-6 carbon atoms, with optional fluorine substitution and optional presence of a single —O— linker. In some embodiments, R²⁵ and R²⁶ are composed of only carbon and hydrogen.

In particular embodiments of Formula (3), the lipophilic crown ether molecule may have the following general formula:

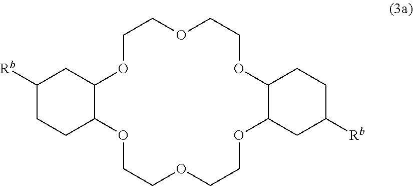

In Formula (3a), R^{b} in each instance is independently selected from one or more of methyl, ethyl, n-propyl, isopropyl, n-butyl, isobutyl, sec-butyl, or t-butyl groups. The two R^{b groups} may be the same or different.

The lipophilic potassium-selective ligand may be, for example, a calixarene

molecule. The lipophilic calixarene molecule may have the following general formula:

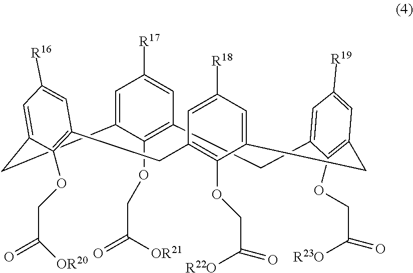

In Formula (4), R¹⁶, R¹⁷, R¹⁸, R¹⁹, R²⁰, R²¹, R²², and R²³ are the same or different and are independently selected from hydrocarbon groups (or more specifically, linear or branched alkyl groups) containing 1-20 carbon atoms, or more particularly, 1-12, 1-6, 1-4, 1-3, 2-20, 2-12, 2-6, 2-4, 3-20, 3-12, or 3-6 carbon atoms, with optional fluorine substitution and optional presence of a single —O— linker. In some embodiments, R¹⁶, R¹⁷, R¹⁸, R¹⁹, R²⁰, R²¹, R²², and R²³ are composed of only carbon and hydrogen. In some embodiments, R¹⁶, R¹⁷, R¹⁸, and R¹⁹ are equivalent to each other, and R²⁰, R²¹, R²², and R²³ are equivalent to each other, but R¹⁶, R¹⁷, R¹⁸, and R¹⁹ are different than R²⁰, R²¹, R²², and R²³.

In particular embodiments of Formula (4), the lipophilic calixarene molecule may

have the following general formula:

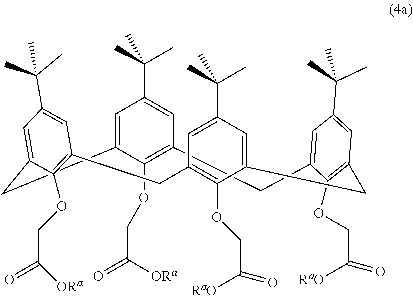

In Formula (4a), R^{a} in each instance is independently selected from one or more of methyl, ethyl, n-propyl, isopropyl, n-butyl, isobutyl, sec-butyl, or t-butyl groups. The R^{a} groups may be the same or different.

The lipophilic sodium-selective and potassium-selective complexing ligands according to any of the formulas provided above can be obtained commercially or synthesized by methods well known in the art. For example, functionalized crown ethers may be prepared as described in F. Nicoli et al., Org. Chem. Front., 8, 5531-5549, 2021, and calixarene molecules may be prepared by base-promoted alkylation of a corresponding phenolic calixarene with chloro- or bromo-acetate derivative, such as described in A. Smirnova et al., European Polymer Journal, 156, 110637 (2021).

In another aspect, the present disclosure is directed to a hydrophobic liquid extractant solution (i.e., “hydrophobic solution”) useful for extracting sodium and potassium from lithium-containing aqueous solutions. The hydrophobic solution is aqueous-insoluble. The hydrophobic solution includes or exclusively (solely) contains: (a) an aqueous-insoluble hydrophobic solvent, (b) a protic ionic liquid of the formula X⁻Y⁺, as described above, and (c) at least one of lipophilic sodium-selective and potassium-selective complexing ligands (i.e., “selective complexing ligands”), as described above.

In the hydrophobic solution, the components (b) and (c) are dissolved in component (a), the aqueous-insoluble hydrophobic solvent. The aqueous-insoluble hydrophobic solvent can be any of the hydrophobic organic solvents known in the art that are substantially or completely immiscible with water or aqueous solutions in general. The aqueous-insoluble hydrophobic solvent is typically a hydrocarbon solvent, which may be non-halogenated (e.g., hexanes, heptanes, octanes, decanes, dodecanes, benzene, toluene, xylenes, kerosene, or petroleum ether), halogenated (e.g., methylene chloride, chloroform, carbon tetrachloride, 1,2-dichlorethane, trichloroethylene, and perchloroethylene), etherified (e.g., diethyl ether or diisopropyl ether), or combination of halogenated and etherified (e.g., bis(chloroethyl)ether and 2-chloroethyl vinyl ether).

Component (b) of the hydrophobic solution includes any one (or more) of the protic ionic liquids described above having any combination of Y⁺ and X⁻ species described above. The protic ionic liquid may more particularly be any protic ionic liquid containing an X⁻ species in combination with Y⁺ being a protic ammonium species. The protic ionic liquid may more particularly be as shown in Formula (1) or (1a), wherein Y⁺ may be any of the protic species described above, or more particularly an ammonium species.

Component (c) of the hydrophobic solution includes at least one of lipophilic sodium-selective and potassium-selective complexing ligands. In some embodiments, component (c) contains a lipophilic sodium-selective complexing ligand. The lipophilic sodium-selective complexing ligand can be, for example, a lipophilic crown ether molecule, such as shown in Formula (3) or (3a). In component (c), the lipophilic sodium-selective complexing ligand may or may not be in the presence of a potassium-selective complexing ligand. In other embodiments, component (c) contains a lipophilic potassium-selective complexing ligand. The lipophilic potassium-selective complexing ligand can be, for example, a lipophilic calixarene molecule, such as shown in Formula (4) or (4a). In component (c), the lipophilic potassium-selective complexing ligand may or may not be in the presence of a sodium-selective complexing ligand. In other embodiments, component (c) contains a lipophilic sodium-selective complexing ligand, such as shown in Formula (3) or (3a), and a lipophilic potassium-selective complexing ligand, such as shown in Formula (4) or (4a).

The protic ionic liquid, i.e., component (b), is present in the hydrophobic solution in any suitable concentration. The concentration of the protic ionic liquid may be, for example, precisely, at least, or up to, for example, 0.01 M, 0.02 M, 0.05 M, 0.1 M, 0.2 M, 0.25 M, 0.3 M, 0.4 M, 0.5 M, 0.6 M, 0.7 M, 0.8 M, 0.9 M, or 1 M or a concentration within a range bounded by any two of the foregoing values, e.g., 0.01-1 M, 0.01-0.5 M, 0.01-0.3 M, 0.01-0.25 M, 0.05-1 M, 0.05-0.5 M, 0.05-0.3 M, 0.05-0.25 M, 0.05-0.2 M, 0.1-1 M, 0.1-0.8 M, 0.1-0.5 M, 0.1-0.25 M, 0.15-1 M, 0.15-0.8 M, 0.15-0.5 M, 0.15-0.3 M, 0.15-0.25 M, 0.2-1 M, 0.2-0.8 M, or 0.2-0.5 M. In some embodiments, the protic ionic liquid is present in the hydrophobic solution in a concentration of about 0.05 M, 0.1 M, or 0.15 M.

The lipophilic sodium-selective complexing ligand in component (c) may be present in the hydrophobic solution in any suitable concentration, such as a concentration of precisely, at least, or up to, for example, 0.01 M, 0.02 M, 0.05 M, 0.1 M, 0.2 M, 0.25 M, 0.3 M, 0.4 M, or 0.5 M, or a concentration within a range bounded by any two of the foregoing values, e.g., 0.01-0.5 M, 0.01-0.3 M, 0.01-0.25 M, 0.05-0.5 M, 0.05-0.3 M, 0.05-0.25 M, 0.05-0.2 M, 0.1-0.5 M, 0.1-0.25 M, 0.15-0.5 M, 0.15-0.3 M, 0.15-0.25 M, or 0.2-0.5 M. In some embodiments, the lipophilic sodium-selective complexing ligand is present in the hydrophobic solution in a concentration of about 0.05 M, 0.1 M, or 0.15 M.

The lipophilic potassium-selective complexing ligand in component (c) may be present in the hydrophobic solution in any suitable concentration, such as a concentration of precisely, at least, or up to, for example, 0.01 M, 0.02 M, 0.05 M, 0.1 M, 0.2 M, 0.25 M, 0.3 M, 0.4 M, or 0.5 M, or a concentration within a range bounded by any two of the foregoing values, e.g., 0.01-0.5 M, 0.01-0.3 M, 0.01-0.25 M, 0.05-0.5 M, 0.05-0.3 M, 0.05-0.25 M, 0.05-0.2 M, 0.1-0.5 M, 0.1-0.25 M, 0.15-0.5 M, 0.15-0.3 M, 0.15-0.25 M, or 0.2-0.5 M. In some embodiments, the lipophilic potassium-selective complexing ligand is present in the hydrophobic solution in a concentration of about 0.05 M, 0.1 M, or 0.15 M.

In some embodiments, the hydrophobic solution contains one or more additional components, such as one or more phase modifiers. Phase modifiers may be, for example, hydrocarbon chain alcohols, including but not limited to, for example, 1-octanol, 1-isooctanol, 1-decanol, 1-dodecanol, or more generally, isomeric branched or linear, primary alcohols that contain both even- and odd-numbered hydrocarbon chains, ranging from C₁ to C₃₀ and/or a mixture of any number of these alcohols or their derivatives including but not limited to esters, ethers, organophosphates, carbonates, and the like. In some embodiments, one or more (or all) such phase modifiers or any additional components is/are excluded from the hydrophobic solution.

In another aspect, the present disclosure is directed to a method for removing (i.e., completely or substantially removing) sodium and/or potassium ions from an alkaline lithium-containing aqueous solution (i.e., aqueous solution). The term “removing,” as used herein, includes at least partially removing, substantially removing, or completely removing. In some embodiments, the alkaline lithium-containing aqueous solution is a lithium-containing brine solution, which is generally naturally alkaline.

The pH of the aqueous solution should be sufficiently high to deprotonate Y⁺ of the ionic liquid. The result is the abstraction of a proton from Y⁺ to form Y, which facilitates the association of the sodium and/or potassium ions with the X⁻ portion of the ionic liquid. Depending on the composition of Y⁺, the pH of the aqueous solution may be, for example, at least or over 7, 8, 9, 10, 11, 12, or 13. The pH of the aqueous solution may also be within a range of any of the foregoing values, e.g., 7-13, 7-12, 7-11, 7-10, 7-9, 8-13, 8-12, 8-11, 8-10, 9-13, 9-12, 9-11, 10-13, or 10-12. In the case where the aqueous solution is lithium-containing brine, the pH is typically high, such as a pH of 13 or 14, and thus, a pH adjuster is generally not included. However, in cases where the aqueous solution is less alkaline, the pH of the aqueous solution can be suitably raised by addition of a base, such as an alkali hydroxide (e.g., NaOH or KOH) or an amine (e.g., ammonia or trimethylamine).

In a first step of the process, i.e., step (i), the lithium-containing aqueous solution is contacted with the aqueous-insoluble hydrophobic solution described above. The hydrophobic solution contains any of the hydrophobic solvents, protic ionic liquids, and lipophilic sodium-selective and/or potassium-selective complexing ligands described above. Any of the concentrations provided above for the protic ionic liquid and complexing ligand(s) may be used in the hydrophobic solution. The term “contacted” or “contacting,” as used herein in reference to contacting of the aqueous and hydrophobic (organic) solutions, generally refers to an intimate mixing of the aqueous and organic phases so as to maximize extraction of the sodium and/or potassium ions from the aqueous phase to the organic phase. Methods of intimately mixing liquids are well known in the art. For example, the aqueous and organic phases may be placed in a container and the container agitated. In some embodiments, the liquids are intimately mixed by subjecting them to vortex mixing. In some embodiments, component (c) of the hydrophobic solution contains only a lipophilic sodium-selective complexing ligand. In other embodiments, component (c) of the hydrophobic solution contains only a lipophilic potassium-selective complexing ligand. In other embodiments, component (c) of the hydrophobic solution contains lipophilic sodium-selective and potassium-selective complexing ligands. In yet other embodiments, the aqueous solution is contacted first with a hydrophobic solution containing only a lipophilic sodium-selective complexing ligand and subsequently contacted with a hydrophobic solution containing only a lipophilic potassium-selective complexing ligand.

In a second step of the extraction process, i.e., step (ii), the two phases are generally separated by means well known in the art, such as by standing followed by decanting, or centrifugation. The foregoing described process amounts to an efficient liquid-liquid extraction process whereby sodium and/or potassium ions in the aqueous solution is/are selectively complexed and removed (extracted) from the aqueous solution into the aqueous-insoluble hydrophobic solvent (organic phase).

The aqueous and organic phases may be used in any suitable volume ratio, wherein the volume of the organic phase is referred to as V_{org} and the volume of the aqueous phase is referred to as V_{aq}. In different embodiments, the V_{org}:V_{aq} (O:A) ratio is precisely, about, or at least, for example, 1:4, 1:3.75, 1:3.5, 1:3.25, 1:3, 1:2.75, 1:2.5, 1:2.25, 1:2, 1:1.75, 1:1.5, 1:1.25, 1:1, 1.25:1, 1.5:1, 1.75:1, 2:1, 2.25:1, 2.5:1, 2.75:1, 3:1, 3.25:1, 3.5:1, 3.75:1, or 4:1, or a ratio within a range bounded by any two of the foregoing ratios, e.g., 1:4-4:1, 1:3-3:1, 1:2-2:1, 1.5:1-1:1.5, 1.25:1-1:1.25, 1:1-4:1, 1:1-3:1, 1:1-2:1, 1:1-1.5:1, 1:1-1:4, 1:1-1:3, 1:1-1:2, and 1:1-1:1.5.

Once the sodium and/or potassium ions are extracted into the hydrophobic solution (organic phase), the sodium and potassium ions may be removed (stripped) from the hydrophobic solution by treating the hydrophobic solution with an acid (typically dissolved in an aqueous stripping solution), wherein the stripping process simultaneously reprotonates Y to regenerate the ionic liquid X⁻Y⁺ in the hydrophobic solution. The pH needed for reprotonation of Y is dependent on the acidity/basicity of Y. In cases where Y is more acidic, the pH used for reprotonation may be, for example, 1, 2, 3, 4, 5, 6, or 7. In cases where Y is less acidic, the pH used for reprotonation may be, for example, 7, 8, or 9. The acid may be an inorganic (mineral) acid or organic acid, any of which may be a strong or weak acid. Some examples of inorganic acids include hydrohalides (i.e., HX, wherein X is typically Cl, Br, or I), sulfuric acid (H₂SO₄), nitric acid (HNO₃), and phosphoric acid (H₃PO₄). Some examples of organic acids include carboxylic acids (e.g., acetic or propionic acid) and sulfonic acids (e.g., triflic acid). In some embodiments, one or more of the foregoing classes or species of acids is excluded from the aqueous stripping solution.

The extraction process is generally capable of achieving a distribution coefficient (D), which may also herein be referred to as an extraction affinity, of at least 1 for sodium or potassium, wherein D is the concentration ratio of sodium or potassium in the organic phase divided by its concentration in the aqueous phase. In some embodiments, a D value of greater than 1 is achieved, such as a D value of at least or above 2, 5, 10, 20, 50, 100, 150, 200, 250, 500, or 1000. The selectivity of the process can be characterized by the separation factor (SF), wherein SF is calculated as the ratio of D for sodium or potassium over the D of any other ion in the aqueous source solution. Selectivity is generally evident in an SF value greater than 1. In some embodiments, an SF (Na/Li or K/Li) value of at least or greater than 2, 5, 10, 20, 50, 100, 150, 200, 250, 300, 350, 400, 450, 500, 600, 700, or 1000 is achieved.

In some embodiments, the resulting aqueous solution substantially removed of sodium and potassium is contacted with a second aqueous-insoluble hydrophobic solution containing a lithium extractant compound to further purify the lithium. The lithium extractant compound is sufficiently lipophilic to extract lithium ions into a hydrophobic phase. The lithium extractant compound is preferably substantially selective in its ability to complex with and transport lithium into a hydrophobic phase over other ions that may be present. Some examples of lipophilic lithium extractant compounds are described in, for example, U.S. Application Pub. No. 2022/0356545, the contents of which are incorporated herein by reference in their entirety. The second hydrophobic solution may also include a non-chelating co-extractant molecule dissolved in the aqueous-insoluble hydrophobic solvent, wherein the non-chelating co-extractant molecule has a single donor group that can form a single coordinate bond with a lithium ion. Alternatively, the aqueous solution substantially removed of sodium and potassium is contacted with a lithium extraction composite for the selective removal of lithium from the aqueous solution. Selective lithium extraction composites are described in, for example, U.S. Pat. No. 11,253,820, the contents of which are incorporated herein by reference in their entirety.

Examples have been set forth below for the purpose of illustration and to describe certain specific embodiments of the invention. However, the scope of this invention is not to be in any way limited by the examples set forth herein.

### EXAMPLES

### Overview

Selective separation and recovery of lithium from terrestrial, geothermal brines, and other sources is a considerable challenge that has received a lot of attention recently. However, the removal of trace contaminants, such as sodium and potassium ions, from brine concentrate is important to facilitate the production of high purity lithium carbonate or hydroxide product. Herein is described a single-step solvent extraction process for purification of lithium ion-containing solutions from trace amounts of sodium (Na⁺) and potassium (K⁺) ions. The process employs a novel protic ionic liquid (IL-1) in combination with selective sodium and/or potassium ion complexing ligands that together permit quick and straight-forward purification of lithium in lithium-containing solutions. Specifically, an ionic liquid (IL-1) containing a highly lipophilic delocalized anion (4-dodecyl-N-((4-dodecylphenyl)sulfonyl) benzenesulfonamide) and tri(2-ethylhexyl)ammonium acts as both an extractant and weak cation exchanger. The selective receptors, tert-butylcalixarene-derived tetraester (L1) or bis(tert-butylcyclohexano)18-crown-6 (L2), bestow additional selectivity to the extraction system. High separation factor values of S_{F} K/Li=390 and S_{F} Na/Li=383 were obtained in a single-stage purification using a synergistic liquid-liquid extraction system with a dual cation extraction.

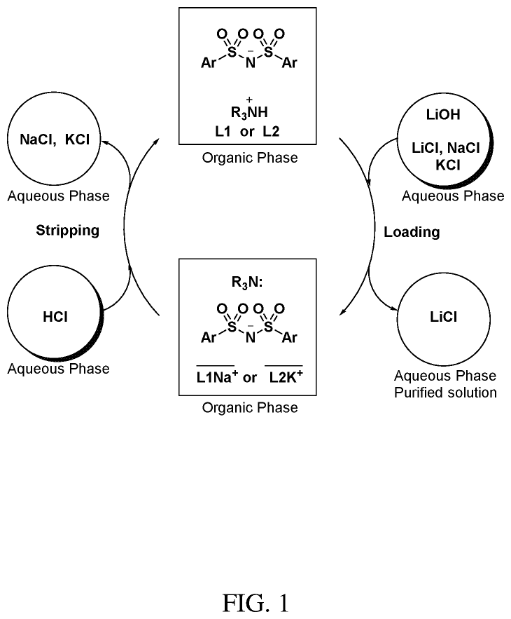

FIG. 1 demonstrates the overall concept of the solvent extraction system for an ion exchange-enabled removal of Na⁺ and K⁺ from Li⁺-containing brine. Na⁺ and K⁺ loading is promoted by the addition of equivalent amounts of high purity lithium hydroxide or carbonate solution, which results in the deprotonation of protic ionic liquid cation. Charge neutrality dictates positive charge compensation by transferring inorganic cations (sodium an potassium in this case) into the organic phase. Next, present in the organic solvent are L1 and L2 neutral donor ligands, which are selective for either Na⁺ or K⁺ cations, respectively, and which preferentially reject lithium and facilitate transport of Na⁺ and K⁺ ions into the organic phase. After phase separation, the loaded organic solution is stripped and regenerated by treatment with an acid solution, which results in the release of Na⁺ and K⁺ ions into the aqueous solution and uptake of protons into the organic phase by protonating the amine moiety that was formed during the neutralization reaction. Overall, acid treatment regenerates the protic ionic liquid that is again ready to be deployed in the purification cycle. Thus, the protic ionic liquid can be recycled for use in one or more additional extractions of sodium and/or potassium ions.

The chemical structure of the IL-1 ionic liquid is provided as follows:

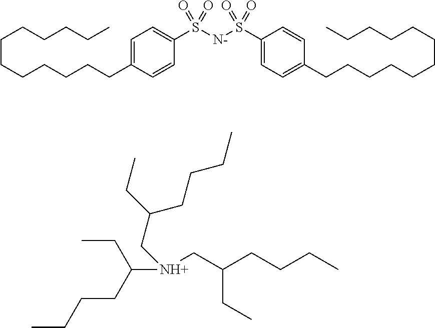 [SMILES: CCCCCCCCCCCCc1ccc(S(=O)(=O)[N-]S(=O)(=O)c2ccc(CCCCCCCCCCCC)cc2)cc1 ; CCCCC(CC)C[NH+](CC(CC)CCCC)C(CC)CCCC]

The chemical structures of synergistic co-extractants L1 and L2 are provided as follows:

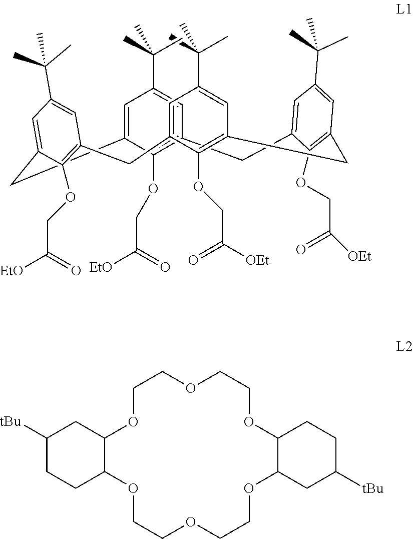

### Experimental Procedures

Extraction/Purification Procedure (1): An aqueous phase consisting of LiOH, LiCl, KCl, NaCl was contacted with equal volume of organic phase. The two phases were contacted at a 1:1 ratio of organic/aqueous by end-over-end rotation in snap-top Eppendorf tubes using a rotating wheel in an air box set at 25.5° C.±0.5° C. Contacts were performed in triplicate with a contact time of at least 1 hour and 10 minutes. Following contacting, the triplicate samples were subjected to centrifuge at 3000×g for 4 minutes at 20° C. to separate the phases. Each triplicate was then subsampled, with 25 μL aliquots of the aqueous phases transferred to individual polypropylene tubes containing 4975 μL of 4% HNO₃ for analysis using ICP-OES. Aqueous samples of stock solutions were also prepared in triplicate and analyzed by ICP-OES for comparison and statistical analysis. The areas found under the observed peaks were used for determining distribution (D) values.

The mean value of the metal measured in triplicate from the aqueous solution after extraction and that of the analyzed initial stock was used to calculate the metal extracted into the organic phase.

[M]_{org}=[M]_{stock}−[M]_{aq} Eq. 1

Mean values were compared to the mean of the measured stock solution through t-test analysis assuming equal variance (using Microsoft Excel Data Package) to determine if significant difference observed.

From these values it is possible to calculate the Distribution coefficient, D, value:

$$D = \frac{{[ M ]}_{org}}{{[ M ]}_{aq}} \qquad Eq . 2$$

And the inter-elemental separation factor, SF according to:

$$SF = \frac{{D}_{M 1}}{{D}_{M 2}} = \frac{X}{Y} \qquad Eq . 3$$

Aqueous Phase: 0.5 M LiCl, 0.1 M LiOH, 0.6 M NaCl and 0.6 M KCl.

Organic Phases:

Solvent only; Isopar L, 1,2-Dichloroethane (DCE), and 10% (v/v) Exxal 13/Isopar L

0.1 M L2 in each of the above solvents

0.1 M L2 combined with Ionic Liquid (IL) in each of the above solvents

IL only in each of the above solvents

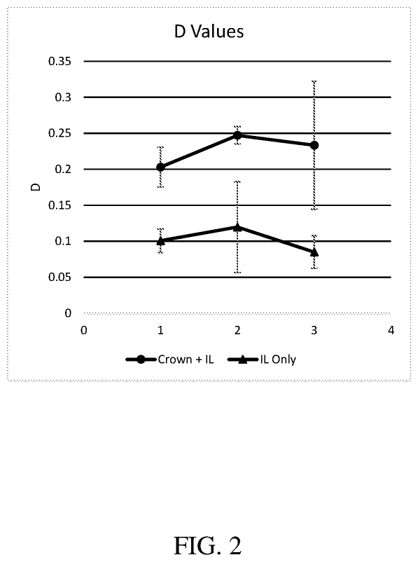

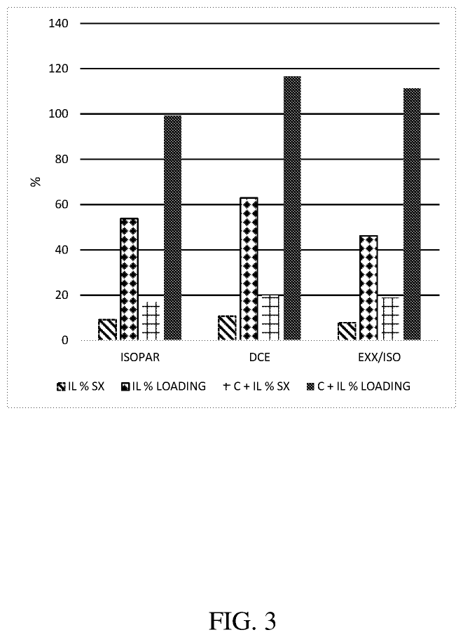

As can be seen from FIGS. 2 and 3, under these extraction conditions, no significant difference was observed in the Li and Na concentrations before and after extraction under any of the organic conditions. For K: no significant difference in K concentration was observed in the solvent only or L2-only extractions (also note that L2 was observed to be insoluble in Isopar L alone). Statistically significant concentrations of K were observed in L2+IL extractions in all solvents with 20% of the available 0.6 M K extracted and 100% ligand loaded (calculated from 0.1 M Ligand).

Due to the zero significant uptake of Li and Na under these high metal conditions, separation factors must be calculated from D values calculated from the Limit of Detection reported for the ICP calibration curve, which is assumed to be maximum [M]_{org}. limit of detection (LoD) for Na is 0.514 ppm and Li is 0.003 ppm.

Extraction/Strip/Purification Procedure (2): An aqueous phase consisting of LiOH, LiCl, KCl, NaCl was contacted with equal volume of organic phase. The two phases were contacted at a 1:1 ratio of organic/aqueous by end-over-end rotation in snap-top Eppendorf tubes using a rotating wheel in an air box set at 25.5° C.±0.5° C. Contacts were performed in triplicate with a contact time of at least 1 hour and 10 minutes. Following contacting, the triplicate samples were subjected to centrifuge at 3000×g for 4 minutes at 20° C. to separate the phases. Each organic phase was then sub-sampled for a stripping stage. Subsamples were contacted with 1 M HCl at a 1:1 ratio of organic/aqueous by end-over-end rotation in snap-top Eppendorf tubes using a rotating wheel in an air box set at 25.5° C.±0.5° C. for 1 hour. The second stripped organic phase was then further subsampled and the strip was repeated under the same conditions. All aqueous phases (post extraction, first strip and second strip) were then sub-sampled, with 200 μL aliquots transferred to individual polypropylene tubes containing 7.8 mL of Li spiked (5 ppm) 4% HNO₃ for analysis using ICP-OES. The areas found under the observed peaks were used for determining distribution (D) values. Aqueous samples of stock solutions, 1 M strip solutions and blank solutions were also prepared in triplicate and analyzed by ICP-OES for comparison and statistical analysis.

The mean value of the metal measured in triplicate from the strip solutions is considered the metal extracted into the organic phase [M]_{org}. Low mean values were compared to the mean of the background solution through t-test analysis assuming equal variance to determine if significant metal present in the organic phase. Metal in the aqueous phase post extraction, [M]_{aq} was calculated from the difference in the measured stock solution [M]_{stock} and that observed in the strip solution, [M]_{org}.

Aqueous Phase 3: 0.15 M LiOH, 0.05 M LiCl 0.1 M KCl and 0.1 M NaCl

Organic Phase 3: 0.05 M L1 and 0.05 M L2 combined with 0.1 M Ionic Liquid (IL-1) in DCE

### Results and Discussion

The protic ionic liquid (IL) used in these experiments contains a highly lipophilic anion and cation pair that still possess weakly acidic character. Anions that have non-nucleophilic and non-coordinating behavior are ideal for the IL due to their much lower affinity toward inorganic cations and resulting better selectivity profile and interfacial behavior. Extraction experiments with alkyl- and aryl-sulfonic acids provided early positive results; however, selectivity profiles and especially interfacial behavior (gelation, precipitation and 3rd phase formation) upon contact with lithium-rich brines did not improve for this class of acids. Next, sulfonimides were studied as possible alternatives to sulfonates. Sulfonimide anions are more delocalized and less nucleophilic compared to the less diffuse sulfonate and carboxylate anions, and thus, more suitable for a spectator role. Additionally, sulfonimides are more acidic and are amenable to the introduction of a variety of functional groups in their structure. 4-dodecyl-N-((4-dodecylphenyl)sulfonyl) benzenesulfonamide (structure shown in IL-1 above) was herein selected as the donor of the anion.

After identifying the anionic part of the protic IL with desirable properties, a plausible class of organic cations was selected. Since the pKa of sulfonimides is generally low, i.e., less than 1, the selection of protonated species will determine the pKa of the protic IL itself. In other words, the conjugated acid of the amine will determine the acidity of the protic IL and not the pKa of the sulfonimide parent acid. Experimentation with several amine classes resulted in the selection of tris (2-ethylhexyl)amine as a viable cation precursor. By combining the solution of the potassium salt of 4-dodecyl-N-((4-dodecylphenyl)sulfonyl) benzenesulfonamide with tris (2-ethylhexyl)ammonium chloride in 1:1 molar ratio in a water/hexane solvent mixture, a hexane solution of protic IL-1 was obtained in high yield. The removal of solvent resulted in a thick honey-like liquid that was readily soluble in non-polar solvents, such hexane, toluene, dichloroethane, and dodecane. Next, the innate cation selectivity of the solvent extraction system with pure protic IL in a non-polar diluent was studied.

Subsequent experiments investigated the extraction ability of hydrophobic solutions containing a non-polar diluent in the presence and absence of synergistic ligands, namely tert-butylcalixarene-derived tetraester (L1) or bis(tert-butylcyclohexano)18-crown-6 (L2), as shown above. L1 has been previously shown to possess high selectivity toward sodium ions in solution. L2 is a more soluble version of the classic 18-crown-6 macrocycle that shows a high selectivity towards the potassium ion.

Extraction experiments using a protic ionic liquid and L1 on the brine containing Na, K and Li ions results in clean extraction of sodium ions with additional extraction of potassium ions and very low uptake of lithium ions. L1 is selective toward sodium ions, but it extracts sodium only in the presence of the IL-1 ion exchanger. The selectivity profile is outstanding and separation factors of S_{F} K/Li=12 and S_{F} Na/Li=984 can be obtained using this system. It is noteworthy that the innate selectivity of the IL-1 extractant was completely reversed by the presence of L1 synergist.

Next, solvent extraction experiments were performed using IL-1 and L2 in dichloroethane solution. As mentioned previously (vide supra), IL-1 has innate selectivity toward potassium ion extraction via ion exchange. This may be due to the fact that the metal ion selectivity is typically inversely correlated with the hydration energy of the cation being extracted into a non-polar medium. The order of decrease in cation hydration energy follows the trend Li⁺>Na⁺>K⁺. Thus, dehydration of potassium ion has the lowest energetic penalty during the phase transfer from aqueous to organic phases. Table 1 (below) shows that IL-1 by itself achieves a high separation factor S_{F} K/Li>819. However, in combination with L1, the separation factor increases dramatically to S_{F} K/Li>1710. This result supports the idea that the addition of L1 and L2 synergistic extractant ligands increases the selectivity of the overall extraction process.

TABLE 1
Separation factors under the extraction conditions in the
presence and absence of synergistic ligand L2 in different
solvents. Notably, L2 increases both selectivity and extraction
ability of the IL-1 based solvent system toward potassium ions.

| | | ISOPAR | DCE | EXXAL/ISOPAR |
|---|---|---|---|---|
| L2 + IL | K/Li SF | >1405 | >1710 | >1614 |
| | K/Na SF | >27 | >33 | >31 |
| IL only | K/Li SF | >697 | >819 | >589 |
| | K/Na SF | >13 | >16 | >11 |

As can be seen from Table 1, under these extraction conditions, both L2+IL and IL alone show completely selective extraction of K ions. In combination with L2, IL shows an increase in D values and percentage extraction which in turn results in a greater overall separation factor but there is no additional increase in selectivity.

To design a system that could selectively extract both sodium and potassium contaminants from lithium-containing brine, extraction experiments were performed utilizing a combination of L1, L2 and IL-1 in an organic diluent. Experiments clearly indicate that the doubly synergistic solvent extraction process permits selective removal of sodium and potassium ions vis-a-vis L1 and L2 ligands respectively. High separation factor values of S_{F} K/Li=390 and S_{F} Na/Li=383 were obtained in this system, which are lower than the performance of IL-1-L1 or IL-1-L2 systems that have only single synergist. One intriguing possibility to overcome the observed behavior and further increased applicability of this novel solvent extraction system is to change the ratios of L1, L2 and IL-1 so that the total amount of L1+L2 is larger than IL-1. In this case, competition between the L1 and L2 for the charge compensated cation that is being transferred into the organic phase could lead to a selective extraction of the single contaminating cation that is present in addition to lithium. In other words, even if there is not enough Na⁺ for L1 or K^{+ for L}2 to be fully complexed, as long as either of these cations is still present in the lithium-rich solution being purified the system will extract only contaminant ions.

### Single Metal Selectivity Tests

In order to assess the differences in selectivity for Li/Na and Li/K aqueous solutions containing just two metals, the solutions containing only Li and Na and only Li and K cation mixtures were tested.

Aqueous Phase 1: 0.1 M LiOH, 0.1 M KCl

Aqueous Phase 2: 0.1 M LiOH, 0.1 M NaCl

Organic Phases:

Solvent only; Isopar L, 1,2-Dichloroethane (DCE), and 10% (v/v) Exxal 13/Isopar L

0.1 M L2 in each of the above solvents

0.1 M L2 combined with Ionic Liquid (IL) in each of the above solvents

IL only in each of the above solvents

### Li/K Extraction

As can be seen from Table 2 (below), no significant Li or K ion uptake was observed in solvent only or L2 only extractions. Under these conditions, statistically significant Li and K were observed by difference in L2+IL extractions in both solvents. For the IL only extraction, third phase or precipitate formation hindered the analysis of DCE samples, but significant Li and K were observed into Exxal/Isopar. Better separation factor was observed in DCE than Exxal/Iso mixture because of a higher D value for K and a lower D value for Li. For the IL only extraction, D values were lower for K and higher for Li, thus resulting in poor separation.

TABLE 2
Potassium extraction with IL-1 only or with IL-1 and L2 in
various solvents

| | | | | | Exxal/ | |
|---|---|---|---|---|---|---|
| | | DCE | SF | | Isopar | SF |
| | Li | K | K/Li | Li | K | K/Li |
| L2 + IL-1 | 0.08 | 15.73 | 333 | 0.16 | 6.04 | 81 |
| D | | | | | | |
| % SX | 4.5 | 94.0 | | 13.89 | 85.80 | |

| % 0.1 M | 97.4 | 91.7 | | |
|---|---|---|---|---|
| Loading | | | | |
| IL Only D | Precipitate | 0.36 | 1.66 | 5 |
| % SX | | 26.77 | 62.35 | |

| | |
|---|---|
| % 0.1 M | 85.7 |
| Loading | |

### Li/Na Extraction

As can be seen from Table 3 (below), statistically significant Li uptake was observed in DCE alone, the same amount observed in the L2 only extraction, around 4%. This is also seen in Exxal/Isopar alone (2.5%) and slightly more in the L2 only extraction (6.5%).

TABLE 3
Sodium extraction with IL-1 only or with IL-1 and L2 in
various solvents.

| | | | | | Exxal/ | |
|---|---|---|---|---|---|---|
| | | DCE | SF | | Isopar | SF |
| | Li | Na | Na/Li | Li | Na | Na/ Li |
| L2 + IL-1 | 0.28 | 3.82 | 14 | 0.46 | 2.53 | 6 |
| D | | | | | | |
| % SX | 21.5 | 79.26 | | 31.4 | 71.7 | |
| [M]_{org}/M | | | | | | |

| % 0.1 M | 95.9 | 100.7 | | |
|---|---|---|---|---|
| Loading | | | | |
| IL Only D | Precipitate | 0.72 | 1.50 | 2 |
| % SX | | 42.17 | 60.07 | |
| [M]_{org}/M | | | | |

| | |
|---|---|
| % 0.1 M | 98.6 |
| Loading | |

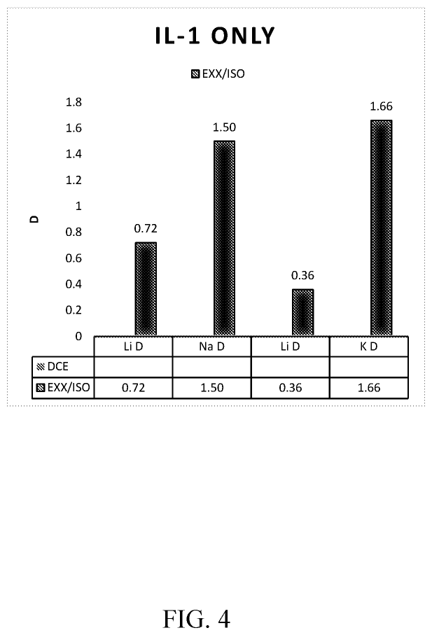

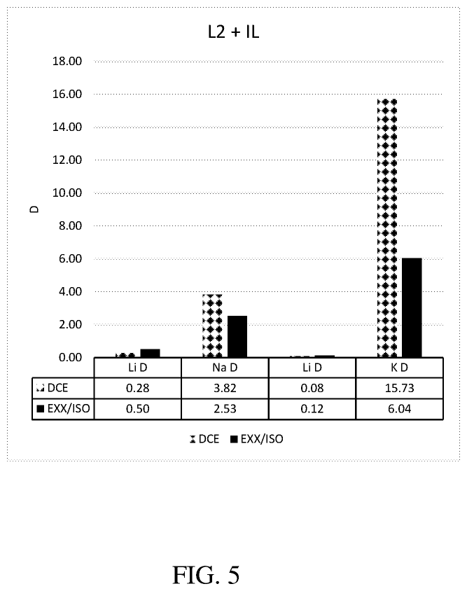

As can be seen from FIGS. 4 and 5, IL-1 is an effective ion exchanger, but selectivity and extractability improve toward Na and K ions with the addition of the L2 synergistic ligand.

### Additional Single Metal Selectivity Tests Using Excess Metal

These metal tests contain both LiOH and LiCl and an excess of either KCl or NaCl.

Aqueous Phase 1: 0.1 M LiOH, 0.1 M LiCl 0.2 M KCl

Aqueous Phase 2: 0.1 M LiOH, 0.1 M LiCl 0.2 M NaCl

Organic Phase: 0.1 M L2 combined with Ionic Liquid (IL-1) in DCE

As can be seen from FIGS. 4 and 5 and Table 4 (below), in the Li/K separation, a small but significant amount of Li is extracted into the organic phase, but D values are higher for K. Using an excess of metal results in lower D values (only half the available 0.2 M of KCl) can be extracted, but, as above, the ligand is 100% loaded (almost exclusively with K).

TABLE 4
Single metal extraction selectivity test results using excess
metal

| | | | SF | | | SF |
|---|---|---|---|---|---|---|
| | Li | K | K/Li | Li | Na | Na/Li |
| L2 + IL D | 0.05 | 0.92 | 17 | 0.19 | 0.66 | 3.5 |
| % SX | 5.09 | 48.0 | | 15.73 | 39.7 | |
| [M]_{org}/M | 0.01 | 0.092 | | 0.029 | 0.073 | |

| | | |
|---|---|---|
| % 0.1 M | 101.4 | 103.2 |
| Loading | | |

As can be seen from FIGS. 4 and 5 and Table 5 (below), in the Li/Na separation, a greater concentration of Li is extracted into the organic phase, and the Li/Na SF is lower. As with the K, excess Na results in lower D values, but the ligand is 100% loaded. However, in this case, the ligand is loaded with around 70% Na and 30% Li.

TABLE 5
Single metal extraction selectivity test results using excess
metal analyzed by difference

| | Li^{#} | K | SF | Li | Na | SF |
|---|---|---|---|---|---|---|
| L2 + IL-1 | 3.0 × 10⁻⁵ | 0.71 | 23438 | 0.035 | 0.726 | 20 |
| D | | | | | | |
| % SX | 0.003 | 41.5 | | 3.46 | 42.1 | |
| [M]_{org}/M | 1.3 × 10⁻⁶ | 0.102 | | 0.007 | 0.083 | |

| | | |
|---|---|---|
| % 0.1 M | 102.5 | 90 |
| Loading | | |

In conclusion, the present experiments successfully demonstrate a synergistic solvent extraction system capable of selectively removing Na⁺ and K⁺ from Li⁺-containing brine. Highly lipophilic protic ionic liquid acts as a cation exchanger, and additional neutral sodium and potassium ion-selective ligands further impart high selectivity toward these ions and rejections of lithium. High separation factor values of S_{F} K/Li=390 and S_{F} Na/Li=383 were obtained in a single-stage purification using a synergistic liquid-liquid extraction system with a dual cation extraction.

While there have been shown and described what are at present considered the preferred embodiments of the invention, those skilled in the art may make various changes and modifications which remain within the scope of the invention defined by the appended claims.

What is claimed is:

1. An ionic liquid composition having the formula: 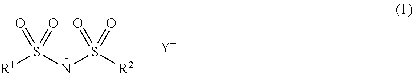
wherein:
R¹ and R² are independently selected from non-fluorinated hydrocarbon groups having 3-30 carbon atoms with optional presence of a single —O— linker; and
Y⁺ is a protonated cation.

2. The ionic liquid of claim 1, wherein Y⁺ is an ammonium species.

3. The ionic liquid of claim 2, wherein the ammonium species has the following formula: 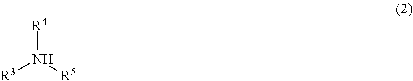
wherein:
R³, R⁴, and R⁵ are independently selected from hydrocarbon groups containing 3-30 carbon atoms.

4. The ionic liquid of claim 1, wherein Y⁺ is a heteroaromatic ring containing at least one nitrogen atom, at least one of which is positively-charged.

5. The ionic liquid of claim 4, wherein the heteroaromatic ring is selected from the group consisting of pyridinium, imidazolium, and pyrrolidinium.

6. The ionic liquid of claim 1, wherein the ionic liquid has the following formula: 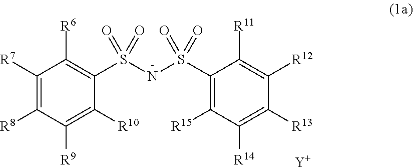
wherein:
R⁶, R⁷, R⁸, R⁹, R¹⁰, R¹¹, R¹², R¹³, R¹⁴, and R¹⁵ are independently selected from H atom and non-fluorinated hydrocarbon groups having 1-30 carbon atoms with optional presence of a single —O— linker, wherein at least one of R⁶, R⁷, R⁸, R⁹, R¹⁰, R¹¹, R¹², R¹³, R¹⁴, and R¹⁵ is a non-fluorinated hydrocarbon group containing 1-30 carbon atoms; and
Y⁺ is a protonated cation.

7. The ionic liquid of claim 6, wherein at least two of R⁶, R⁷, R⁸, R⁹, R¹⁰, R¹¹, R¹², R¹³, R¹⁴, and R¹⁵ are non-fluorinated hydrocarbon groups containing 1-30 carbon atoms.

8. The ionic liquid of claim 6, wherein Y⁺ is an ammonium species.

9. The ionic liquid of claim 8, wherein the ammonium species has the following formula: 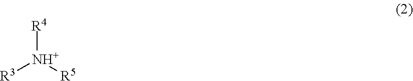
wherein:
R³, R⁴, and R⁵ are independently selected from hydrocarbon groups containing 3-30 carbon atoms.

10. The ionic liquid of claim 6, wherein Y⁺ is a heterocyclic ring containing at least one nitrogen atom, at least one of which is positively-charged.

11. The ionic liquid of claim 10, wherein the heterocyclic ring is selected from the group consisting of pyridinium, imidazolium, piperidinium, and pyrrolidinium.

12. A method for selectively removing sodium or potassium from an alkaline lithium-containing aqueous solution, the method comprising:
(i) contacting said alkaline lithium-containing aqueous solution with a hydrophobic solution comprising: (a) an aqueous-insoluble hydrophobic solvent, (b) a protic ionic liquid of the formula X⁻Y⁺, wherein X⁻ is a conjugate base of a superacid and Y⁺ is a protonated cation, and (c) at least one of lipophilic sodium-selective and potassium-selective complexing ligands, wherein the contacting step results in selective complexation with and removal of sodium and/or potassium ions from the lithium-containing aqueous solution into the hydrophobic solution along with simultaneous abstraction of a proton from Y⁺ to form Y; and
(ii) separating the aqueous solution from the hydrophobic solution.

13. The method of claim 12, further comprising stripping sodium and potassium ions from the hydrophobic solution by treating the hydrophobic solution with an acid, wherein the stripping process simultaneously reprotonates Y to regenerate the ionic liquid X⁻Y⁺ in the hydrophobic solution.

14. The method of claim 12, wherein the alkaline lithium-containing aqueous solution is a lithium-containing brine solution.

15. The method of claim 12, wherein the protic ionic liquid has the formula: 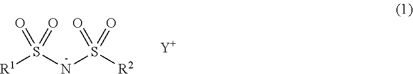
wherein:
R¹ and R² are independently selected from non-fluorinated hydrocarbon groups having 3-30 carbon atoms with optional presence of a single —O— linker; and
Y⁺ is a protonated cation.

16. The method of claim 12, wherein Y⁺ is an ammonium species.

17. The method of claim 16, wherein the ammonium species has the following formula: 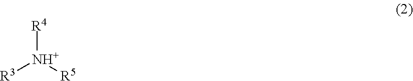
wherein:
R³, R⁴, and R⁵ are independently selected from hydrocarbon groups containing 3-30 carbon atoms.

18. The method of claim 12, wherein Y⁺ is a heterocyclic ring containing at least one nitrogen atom, at least one of which is positively-charged.

19. The method of claim 18, wherein the heterocyclic ring is selected from the group consisting of pyridinium, imidazolium, piperidinium, and pyrrolidinium.

20. The method of claim 12, wherein the ionic liquid has the following formula: 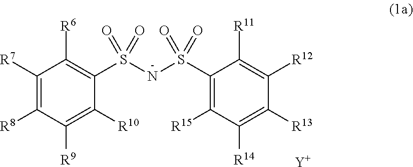
wherein:
R⁶, R⁷, R⁸, R⁹, R¹⁰, R¹¹, R¹², R¹³, R¹⁴, and R¹⁵ are independently selected from H atom and non-fluorinated hydrocarbon groups having 1-30 carbon atoms with optional presence of a single —O— linker, wherein at least one of R⁶, R⁷, R⁸, R⁹, R¹⁰, R¹¹, R¹², R¹³, R¹⁴, and R¹⁵ is a non-fluorinated hydrocarbon group containing 1-30 carbon atoms; and
Y⁺ is a protonated cation.

21. The method of claim 20, wherein at least two of R⁶, R⁷, R⁸, R⁹, R¹⁰, R¹¹, R¹², R¹³, R¹⁴, and R¹⁵ are non-fluorinated hydrocarbon groups containing 1-30 carbon atoms.

22. The method of claim 20, wherein Y⁺ is an ammonium species.

23. The method of claim 20, wherein Y⁺ is a heteroaromatic ring containing at least one nitrogen atom, at least one of which is positively-charged.

24. The method of claim 12, wherein the lipophilic sodium-selective complexing ligand is a lipophilic crown ether molecule and is present in the hydrophobic solution.

25. The method of claim 12, wherein the lipophilic potassium-selective complexing ligand is a calixarene molecule and is present in the hydrophobic solution.
